(19) United States
(12) Patent Application Publication
(10) Pub. No.: US 2018/0117096 A1
(43) Pub. Date: May 3, 2018

(54) METHOD FOR REDUCING METHANE PRODUCTION IN A RUMINANT ANIMAL

(71) Applicant: DUPONT NUTRITION BIOSCIENCES APS, Copenhagen (DK)

(72) Inventors: CLAUDETTE BERGER, MENNECY (FR); ABDERZAK LETTAT, VENISSIEUX (FR); CECILE MARTIN, SAINT SATURNIN (FR); PIERRE NOZIERE, TALLENDE (FR)

(21) Appl. No.: 15/855,664

(22) Filed: Dec. 27, 2017

Related U.S. Application Data
(63) Continuation of application No. 14/113,927, filed on Jan. 7, 2014, now abandoned.
(60) Provisional application No. 61/479,222, filed on Apr. 26, 2011.

(51) Int. Cl. A61K 35/74; A23K 10/16; A23K 50/10

(52) U.S. Cl. CPC A61K 35/74; Y02P 60/56; A23K 50/10; A23K 10/16

(57) ABSTRACT

A method for reducing methane production in a ruminant animal comprising the step of administering to said ruminant animal an effective amount of at least one strain of bacterium of the genus Propionibacterium.

### FIELD OF THE INVENTION

The present invention relates to a method of reducing methane production in a ruminant animal.

### BACKGROUND OF THE INVENTION

According to a recent UN report, cattle-rearing generates more global warming greenhouse gases, as measure in CO₂ equivalent, than transportation, and smarter production methods, including improved animal diets to reduce enteric fermentation and consequent methane emissions, are urgently needed.

Seeking methods of reducing methane production in ruminant animals therefore represents a major challenge. The present invention seeks to solve this unmet need in the industry.

A key advantage of the present invention is that it provides a method to reduce methane production in a ruminant without modifying the diet nor introducing methane producer blocking agents. In other words a key innovation of the invention is the fact that there is no change versus a standard/normal diet of the ruminants. This is in contrast to the prior art which required a change in the diet and/or the introduction of some agent to block the methane-producing bacteria.

### STATEMENTS OF THE INVENTION

According to a first aspect of the present invention there is provided a method for reducing methane production in a ruminant animal comprising the step of administering to said ruminant animal an effective amount of at least one strain of bacterium of the genus Propionibacterium.

Preferably said strain of bacterium belongs to the species Propionibacterium jensenii, Propionibacterium acidipropionici, Propionibacterium freudenreichii or Propionibacterium freudenreichii ssp shermanii.

More preferably said strain belongs to the species Propionibacterium jensenii.

Even more preferably the strain is Propionibacterium jensenii P63.

Preferably the method further comprises the step of administering to said ruminant animal an effective amount of at least one strain of bacterium of the genus Lactobacillus.

Preferably said strain of bacterium of the genus Lactobacillus belongs to the species L. helveticus, L. amylovorus, L. curvatus, L. cellobiosus, L. amylolyticus, L. alimentarius, L. aviaries, L. crispatus, L. curvatus, L. gallinarum, L. hilgardii, L. johnsonii, L. kefiranofaecium, L. kefiri, L. mucosae, L. panis, L. pentosus, L. pontis, L. zeae, L. sanfranciscensis, L. paracasei, L. casei, L. acidophilus, L. buchnerii, L. farciminis, L. rhamnosus, L. reuteri, L. fermentum, L. brevis, L. lactis, L. plantarum, L. sakei or L. salviarium.

More preferably the strain of bacterium of the genus Lactobacillus belongs to the species L. rhamnosus, L. farciminis, L. buchnerii, L. helveticus, L. amylovorus, L. curvatus, L. cellobiosus, L. amylolyticus, L. alimentarius, L. aviaries, L. crispatus, L. curvatus, L. gallinarum, L. hilgardii, L. johnsonii, L. kefiranofaecium, L. kefiri, L. mucosae, L. panis, L. pentosus, L. pontis, L. zeae or L. sanfranciscensis.

More preferably the strain of bacterium of the genus Lactobacillus belongs to the species L. plantarum or L. rhamnosus.

Even more preferably the strain of the bacterium of the genus Lactobacillus is L. plantarum Lp115 or L. rhamnosus Lr32.

Preferably at least one strain of bacterium of the genus Propionibacterium and the at least one strain of the bacterium of the genus Lactobacillus are administered as a mixture.

More preferably said mixture of at least two strains of bacteria is a mixture of at feast one strain of L. plantarum or L. rhamnosus and at least one strain of Propionibacterium jensenii.

Even more preferably said mixture is a mixture of L. plantarum Lp115 or L. rhamnosus Lr32 and Propionibacterium jensenii P63.

Preferably the at least one strain of bacteria is inactivated.

Preferably said effective amount of at least one strain of bacterium is administered to said ruminant animal by supplementing food intended for said animal with said effective amount of at least one strain of bacterium.

In one embodiment the method additionally improves the digestibility of the supplementing food.

In one embodiment the method additionally increases milk fat production by the ruminant animal.

In one embodiment the method additionally increases milk lactose production by the ruminant animal.

In one embodiment the method additionally improves the body weight of the ruminant animal.

Preferably said ruminant animal is selected from the members of the Ruminantia and Tylopoda suborders.

Preferably said ruminant animal is selected from the members of the Antilocapridae, Bovidae, Cervidae, Girraffidae, Moschidae, Tragulidae families.

Preferably said ruminant animal is a cattle, goat, sheep, giraffe, bison, yak, water buffalo, deer, camel, alpaca, llama, wildebeest, antelope, pronghorn or nilgai.

More preferably said ruminant animal is a cattle or sheep.

Even more preferably said ruminant animal is a cattle.

According to a second aspect of the present invention there is provided a feed supplement for a ruminant animal for reducing methane production comprising at least one strain of bacterium the genus Propionibacterium.

Preferably the feed supplement further comprises at least one strain of bacterium of the genus Lactobacillus.

According to a third embodiment of the present invention there is provided a feed for a ruminant animal, wherein said feed is supplemented with a feed supplement according to the present invention.

According to a fourth aspect of the present invention there is provided a method for reducing methane production by a ruminant animal, said method comprising the step of administering to said animal a feed supplement according to the present invention.

According to a fifth aspect of the present invention there is provided a method for increasing milk fat production by a ruminant animal, said method comprising the step of administering to said animal a feed supplement according to the present invention.

According to a sixth aspect of the present invention there is provided a method for increasing milk lactose production by a ruminant animal, said method comprising the step of administering to said animal a feed supplement according to the present invention.

According to a seventh aspect of the present invention there is provided a method for increasing the body weight of a ruminant animal, said method comprising the step of administering to said animal a feed supplement according to the present invention.

### DETAILED DESCRIPTION OF THE INVENTION

The present invention relates to a method for reducing methane production by a ruminant animal. Surprisingly and unexpectedly, the inventors have shown that certain bacteria possess the property of reducing methane production in ruminant animals. These bacteria belong to the genus Propionibacterium, optionally administered with at least one strain of the genus Lactobacillus. The invention therefore relates to a method for reducing methane production in a ruminant animal comprising the step of administering to said ruminant animal an effective amount of at least one strain of bacterium of the genus Propionibacterium, optionally with a bacterium of genus Lactobacillus.

A ruminant is a mammal of the order Artiodactyla that digests plant-based food by initially softening it within the animal's first stomach, then regurgitating the semi-digested mass, now known as cud, and chewing it again. The process of rechewing the cud to further break down plant matter and stimulate digestion is called “ruminating”. Ruminants have a stomach with four chambers, namely the rumen, reticulum, omasum and abomasum. In the first two chambers, the rumen and the reticulum, the food is mixed with saliva and separates into layers of solid and liquid material. Solids clump together to form the cud, or bolus. The cud is then regurgitated, chewed slowly to completely mix it with saliva, which further breaks down fibers. Fiber, especially cellulose, is broken down into glucose in these chambers by symbiotic bacteria, protozoa and fungi. The broken-down fiber, which is now in the liquid part of the contents, then passes through the rumen into the next stomach chamber, the omasum, where water is removed. The food in the abomasum is digested much like it would be in the human stomach. It is finally sent to the small intestine, where the absorption of the nutrients occurs.

Almost all the glucose produced by the breaking down of cellulose is used by the symbiotic bacteria. Ruminants get their energy from the volatile fatty acids produced by the bacteria, namely lactic acid, propionic acid and butyric acid.

The rumen is the major source of methane production in ruminants.

Examples of ruminants are listed above. However, preferably the bacteria is used as an additive for foodstuffs for domesticated livestock such as cattle, goats, sheep and llamas. The present invention is particularly useful in cattle.

By “administer”, is meant the action of introducing at least one strain of bacterium according to the invention into the animal's gastrointestinal tract. More particularly, this administration is an administration by oral route. This administration can in particular be carried out by supplementing the feed intended for the animal with said at least one strain of bacterium, the thus supplemented feed then being ingested by the animal. The administration can also be carried out using a stomach tube or any other means making it possible to directly introduce said at least one strain of bacterium into the animal's gastro-intestinal tract.

By “effective amount”, is meant a quantity of bacteria sufficient to allow improvement, i.e. reduction in the amount of methane production in comparison with a reference. Within the meaning of the present invention, the methane reductive effect can be measured in the rumen with an artificial rumen system, such as that described in T. Hano., J. Gen. Appl. Microbiol., 39, 35-45)1993) or by in vivo oral administration to ruminants.

This effective amount can be administered to said ruminant animal in one or more doses.

By “at least one strain”, is meant a single strain but also mixtures of strains comprising at least two strains of bacteria.

By “a mixture of at least two strains”, is meant a mixture of two, three, four, five, six or even more strains.

When using a mixture of strains the proportions can vary from 1% to 99%, more advantageously from 25% to 75% and even more advantageously approximately 50% for each strain. In a mixture comprising more than two strains, the strains are preferentially present in substantially equal proportions in the mixture.

The strains of the genus Propionibacterium are in particular selected from strains of the species Propionibacterium jensenii, Propionibacterium acidipropionici, Propionibacterium freudenreichii and Propionibacterium freudenreichii ssp shermanii. A particular strain of the species Propionibacterium jensenii according to the invention is the strain Propionibacterium jensenii P63, deposited under the Budapest Treaty on 15 Jan. 2009, in the Deutsche Sammlung von Mikroorganismen and Zellkulturen GmbH (DSMZ, Inhoffenstr. 7 B, D-38124 Braunschweig, Germany) under number DSM22192 by Danisco Deutschland GmbH (Bush-Johannsen-Str. 1, 25899 Niebull, Germany).

According to an embodiment of the invention, the strains of the genus Lactobacillus are in particular selected from the species L. paracasei, L. casei, L. acidophilus, L. buchnerii, L. farciminis, L. rhamnosus, L. reuteri, L. brevis, L. fermentum, L. lactis, L. plantarum, L. sakei, L. salviarium, L. helveticus, L. amylovorus, L. curvatus, L. cellobiosus, L. amylolyticus, L. alimentarius, L. aviaries, L. crispatus, L. curvatus, L. gallinarum, L. hilgardii, L. johnsonii, L. kefiranofaecium, L. kefiri, L. mucosae, L. panis, L. pentosus, L. pontis, L. zeae or L. sanfranciscensis.

Examples of mixtures of strains of bacteria according to the invention are in particular a mixture comprising at least two strains of the genus Lactobacillus and at least one strain of the genus Propionibacterium.

More preferably the strain of bacterium of the genus Lactobacillus belongs to the species L. plantarum or L. rhamnosus.

A particular strain of the species L. plantarum according to the invention is the strain L. plantarum Lp115, deposited under the Budapest Treaty on 9 Feb. 2009, in the Deutsche Sammlung von Mikroorganismen and Zellkulturen GmbH (DSMZ, Inhoffenstr. 7 B, D-38124 Braunschweig, Germany) under number DSM22266 by Danisco Deutschland GmbH (Bush-Johannsen-Str. 1, 25899 Niebull, Germany).

A particular strain of the species L. rhamnosus according to the invention is the strain L. rhamnosus L32, deposited under the Budapest Treaty on 15 Jan. 2009, in the Deutsche Sammlung von Mikroorganismen and Zellkulturen GmbH (DSMZ, Inhoffenstr, 7 B, D-38124 Braunschweig, Germany) under number DSM22193 by Danisco Deutschland GmbH (Bush-Johannsen-Str. 1, 25899 Niebull, Germany).

According to a particular embodiment, the methods according to the invention also comprise the step of administering other microorganisms, said microorganisms being selected from the group comprising in particular the lactic bacteria, probiotic microorganisms, yeasts and fungi (for example Penicillium and Geotrichum).

According to one embodiment, the additional microorganism is a bacteria of the genus Pediococcus, and particularly Pediococcus acidilactici.

According to an embodiment of the invention, the strains of bacteria are inactivated before their administration to the ruminant animal. The inactivation makes it possible to significantly reduce the microorganisms' ability to reproduce without significantly affecting their enzymatic activity. Typically, following the inactivation process, the number of microorganisms capable of reproducing is reduced by a factor greater than X, X being selected from the following values; 10.sup.4, 10.sup.5, 10.sup.6, 10.sup.7, 10.sup.8, 10.sup.9, 10.sup.10 and 10.sup.11.

Typically, the microorganisms can be inactivated by a heat shock treatment. For example, the microorganisms can be exposed to temperatures comprised between 40.degree. C, and 70.degree. C. The duration of the heat shock treatment will depend on the chosen temperature and the microorganism to be inactivated. For example, the inactivation method can be carried out over a period of time comprised between 15 minutes and 96 hours. For example also, the microorganisms can be exposed to temperatures comprised between 60.degree. C. and 70.degree. C. for a period of time comprised between 20 and 40 hours.

Other techniques can be used to inactivate the microorganisms, such as for example ionization or photoinactivation (inactivation by light). The microorganisms can also be inactivated by keeping them for long periods at a temperature or humidity level which is not compatible with their viability.

The inactivation of the strains of bacteria according to the invention has the consequence of preventing the multiplication and development of the bacteria while preserving their methane-reducing properties. Moreover, the inactivation of the strains means that the strains will not enter into competition with the fibrolytic, cellulolytic and amylolytic intestinal flora, while releasing their enzyme content into the medium.

According to the present invention, said effective amount of said at least one strain of bacterium is typically comprised between 10.sup.5 CFU and 10.sup.13 CFU per animal and per day, particularly between 10.sup.7 CFU and 10.sup.12 CFU per animal and per day, more particularly between 10.sup.8 CFU and 10.sup.11 CFU per animal and per day, even more particularly approximately 10.sup.10 CFU per animal and per day. When the bacteria are inactivated, the quantities described previously are calculated before inactivation.

The bacterium of the present invention can be administered, for example, as the fermentation mixture, bacterium-containing culture solution, the bactenum-containing supernatant or the bacterial product of a culture solution.

The bacterium may be administered to the ruminant in one of many ways. The culture can be administered in a solid form as a veterinary pharmaceutical, may be distributed in an excipient, preferably water, and directly fed to the animal, may be physically mixed with feed material in a dry form or the culture may be formed into a solution and thereafter sprayed onto feed material. The method of administration of the culture to the animal is considered to be within the skill of the artisan.

When used in combination with a feed material, the feed material is preferably grain, hay/silage/grass/based. Included amongst such feed materials are corn, dried grain, alfalfa, any feed ingredients and food or feed industry by-products as well as bio-fuel industry by-products and corn meal and mixtures thereof.

The bacterium of the novel process may optionally be admixed with a dry formulation of additives including but not limited to growth substrates, enzymes, sugars, carbohydrates, extracts and growth promoting micro-ingredients. The sugars could include the following: lactose; maltose; dextrose; malto-dextrin; glucose; fructose; mannose; tagatose; sorbose; raffinose; and galactose. The sugars range from 50-95%, either individually or in combination. The extracts could include yeast, or dried yeast fermentation solubles ranging from 5-50%. The growth substrates could include: trypticase, ranging from 5-25%; sodium lactate, ranging from 5-30%; and, Tween 80, ranging from 1-5%. The carbohydrates could include mannitol, sorbitol, adonitol and arabitol. The carbohydrates range from 5-50% individually or in combination. The micro-ingredients could include the following: calcium carbonate, ranging from 0.5-5.0%; calcium chloride, ranging from 0.5-5.0%; dipotassium phosphate, ranging from 0.5-5.0%; calcium phosphate, ranging from 0.5-5.0%; manganese proteinase, ranging from 0.25-1.00%; and, manganese, ranging from 0.25-100%.

The time of administration is not crucial so long as the reductive effect on methane production is shown. As long as the feed is retained in the rumen, administration is possible at any time. However, since the bacterium is preferably present in the rumen at about the time methane is produced, the bacterium is preferably administered with or immediately before feed.

According to one embodiment of the invention, the present invention improves the digestibility of the supplementing food. The digestibility of the fibres is considered “improved” if the fibres are better digested by the animal in the presence of said at least one strain of bacterium, in a non-limitative manner, methods which can be used to measure the digestibility of the fibres are the methods of measuring the final fermentation products. For instance, measurement of lactic acid, for example by an enzymatic colorimetric method, and measurement of volatile fatty acids (VFAs), for example by gas chromatography as described by Jouany JP and Senaud J in Reprod Nutr Dev. 1982; 22(5):735-52, are suitable. Thus, using these methods, a person skilled in the art is able to compare digestibility in the presence and in the absence of the strains of bacteria according to the invention.

Thus, in a particular embodiment of the invention, said effective amount of at least one strain of bacterium is administered to a ruminant animal by supplementing a feed intended for said animal with said effective amount of at least one strain of bacterium. By “supplementing”, within the meaning of the invention, is meant the action of incorporating the effective amount of bacteria according to the invention directly into the feed intended for the animal. Thus, the animal, when feeding, ingests, the bacteria according to the invention which can then act to increase e.g. the digestibility of the fibres and/or cereals contained in the animal's feed.

Thus, another subject of the invention relates to a feed supplement for a ruminant animal comprising at least one strain of Propionibacterium bacterium and optionally at least one strain of bacteria of the genera Lactobacillus.

In one embodiment, the present invention improves the body weight of the ruminant animal. Thus, this method allows the ruminant animal to derive greater benefit in terms of energy from feed based on e.g. fibres and cereals, and as a result, starting from the same calorie intake, to increase the energy available to its metabolism. This is advantageous for the livestock farmer who can thus optimize the cost of the feed. In fact, he can either reduce the animal's feed for the same energy intake or reduce the quantity of starchy cereals and replace it with less expensive fibre-rich fodder, which allows him to make a financial saying.

By “increasing body” we include that the bacterium increases the body weight by at least 1%, 2%, 3%, 4% or 5-10%, in comparison to a reference sample.

In one embodiment the method increases milk fat production by the ruminant animal. This also represents a substantial economic benefit.

Milk solid components include protein, fat, lactose, and minerals. Milk protein has economic value because, for example, higher protein leads to higher cheese yields. Furthermore, in recent years, consumers have become increasingly concerned about the effects of dietary fat consumption on their health. Low fat milk and low fat cheese have become popular. In many countries, including the United States, the payment for milk shipped to cheese plants has changed to a system based on both protein and fat content from one based on milk fat. This market trend increases the emphasis on milk protein. However, milk fat continues to be an important component in some markets where it is used to make ice cream and butter. In these markets, a premium of $2 per pound is paid for milk fat.

By “increasing milk fat production” we include that the bacterium increases milk fat production by at least 1%, 2%, 3%, 4%, or 5-10% of the weight of the product in comparison to a reference sample.

In another embodiment of the invention the method increases milk lactose production. This also repmsents a substantial economic benefit.

Lactose is a disaccharide sugar that is found most notably in milk and is farmed from galactose and glucose. Lactose makes up around 2˜8% of milk (by weight), although the amount varies among species and individuals. It is extracted from sweet or sour whey.

Food industry applications, both of pure lactose and lactose-containing dairy by-products, have markedly increased since the 1960s. For example, its bland flavour has lent to its use as a carrier and stabiliser of aromas and pharmaceutical products. Purified lactose can also be used as high calorie diet additive.

By “increasing milk lactose production” we include that the bacterium increases milk lactose production by at least 1%, 2%, 3%, 4% or 5-10% of the weight of the product, in comparison to a reference sample.

The invention will now be further described by way of Examples, which are meant to serve to assist one of ordinary skill in the art in carrying out the invention and are not intended in any way to limit the scope of the invention.

### EXAMPLES

The experiment was conducted at the animal experimental facilities of the INRA's Herbivores Research Unit (Saint-Genès Champanelle, France). Procedures on animals were in accordance with the guidelines for animal research of the French Ministry of Agriculture and all other applicable national and European guidelines and regulations for experimentation with animals (see http://www2.vet-lyon.fr/ens/expa/acc_regl.html for details). The experiment was approved by the Auvergne regional ethic committee for animal experimentation, approval number CE20-09. “DFM” refers to bacterial direct-fed microbial. “SARA” refers to subacute ruminal acidosis. “DIM” refers to days in milk. DMI refers to dry matter intake. “DM” refers to dry matter. “OM” refers to organic matter. “ADF” refers to acid detergent fiber. “NDF” refers to neutral detergent fiber. “GE” refers to gross energy. “CP” refers to crude protein. “CMV” refers to complement of vitamins and minerals. “VFA” refers to volatile fatty acids. “BW” refers to body weight. “VFA” refers to volatile fatty acids.

### Animals, Diets and Experimental Procedure

Eight lactating Holstein cows (2 primi- and 8 multiparous) fitted with ruminal cannulas and were allocated to 2 groups of 4 animals differing by the nature of their diet (high starch diet (HSD) vs. low starch diet (LSD); Table 1) in order to induce two subacute ruminal acidosis situations with propionate and butyrate as the main fermentation end products. At the initiation of the experiment, DIM averaged 76±19 and 67±22 d (mean±SD), BW was 587±20 and 596±43 kg and milk production was 27±3 and 28±6 kg/d for the cows on the HSD and LSD, respectively. Cows in each group were randomly assigned to four treatments in a 4×4 Latin square design. The treatments were 1) control without DFM (C), 2) Propionibacterium P63 (P), 3) Lactobacillus plantarum strain 115 plus P (Lp+P) and 4) Lb. rhamnosus strain 32 plus P (Lr+P). To ensure an entire consumption of the DFM, the 4 treatments were mixed with a small portion of concentrate (sampled from their diet) and offered before the morning feeding. Cows on the DFM treatments received 10¹⁰ CFU/d of each strain whereas control cows received only carrier (lactose). To minimize the carryover from period-to-period, on the last day of period 1, 2 and 3, the rumen of each cow was manually emptied and the ruminal contents were placed into the rumen of the next cow within the square that was to receive that treatment. Thus, each cow started the period with ruminai contents corresponding to the same treatment it was fed (Beauchemin et al., 2003).

Diets were formulated at the beginning of the experiment from milk yield to meet the requirements for maintenance and milk production of the cow (INRA, 1989) and were readjusted each experimental period assuming a monthly decrease in milk production of 10%. Moreover, the diets were free of antibiotics, chemical buffers and yeast to avoid any interfering with DFM effect. Concentrates were top-dressed on the silages, to favor acidosis induction by its rapid ingestion. Each experimental period lasted 1 month, and the 2 last weeks were used for animal performances (wk 3) and ruminal parameters measurements (wk 4).

### Lactation Study During Wk 3

Intake and feed analysts To calculate DMI, feed intakes and orts were recorded weekly on five consecutive days throughout the entire experiment. DM content was measured at 103° C. for 24 h twice weekly for silages and hay, and once a week for concentrates. During wk 3 of each experimental period, daily samples of each ingredient (exception made for urea, cane molasses and CMV that were only sampled on the first period and for which the composition was thought to be stable over time) were taken and stored at 4° C. for concentrates and hay, and at −20° C. for corn and grass silage pending chemical composition determination. At the end of the experiment, dried (60° C. for 48 h) silages and hay samples, as well as pooled concentrate samples were ground to pass through 1-mm screen then analyzed for OM, NDF, ADF, starch, nitrogen, GE and ether extract. Moreover, fresh samples of concentrate and forages were freeze-dried and ground (1 mm) for fatty acids analysis. All the feedstuff analyses were performed as previously reported (Martin et al., 2008). OM by ashing at 550° C. for 6 h (AOAC, 1990), nitrogen content of feed was analyzed by the Kjeldahl procedure. The NDF and ADF contents were determined by sequential procedures after pretreatment with amylase and were expressed inclusive of residual ash (Van Soest et al., 1991). Polarimetric method was used for starch quantification (AFNOR, 1985) and AOAC procedure (1997) was used for ether extract analysis. The GE content was determined using an adiabatic bomb calorimeter (IKA C200, Bioritech, Guyancourt, France).

Milk yield and composition Cows were milked twice daily (0730 and 1500 h) throughout the entire experiment and milk was analyzed twice a wk (non-consecutive days) for fat, protein, lactose and somatic cell counts (SCC) using standard procedure (AOAC, 1997).

Diet Digestibility During wk 3, feces and urine were collected on 6 consecutive days were used for total tract digestibility determination. Each day, feces were collected, weighed and mixed. 1% aliquot was used for DM determination at 103° C. for 24 h, and 0.5% aliquot was collected and pooled within a Latin square by treatment. At the end of the experiment pooled samples were dried (60° C. for 72 h) then ground (1-mm screen) then analysed for OM, NDF, ADF, starch as described previously.

Methane emissions They were determined during the same period of digestibility measurement by using the sulfur hexafluoride (SF₆) tracer technique (Johnson et al., 2007) as previously described (Martin et al., 2008). Permeation rate of SF₆ from the tubes was 1.503±0.145 mg/d. At wk-2 of each experimental period (1 wk before gas analysis), each cow was intraruminally dosed with a calibrated permeation tube. Representative breath samples from each animal were sampled in preevacuated (91.2 kPa) yoke-shaped polyvinyl chloride collection devices (˜2.5 L) by means of capillary and Teflon tubing fitted to a halter. The collection devices were changed every 24 h before the morning feeding. Background concentrations of SF₆ and CH₄ were also measured in ambient air samples collected every day in the shed during the same 6-d breath sampling period. The devices containing the samples were immediately transported to the laboratory and over-pressured with nitrogen gas prior to SF₆ and CH₄ analyses by gas chromatography. Daily CH₄ production from each animal was calculated according to Martin et al. (2008), using the known permeation rate of SF₆ and the concentrations (above the background) of SF₆ and CH₄ in the breath samples:

CH₄ (g/d)=SF₆ permeation rate (g/d)×[CH₄]/[SF₆]

For gas analysis in breath and ambient air, we used a gas chromatograph (CP-9003, Varian-Chrompack, Les Ulis, France) fitted with an electron capture detector (Autosystem XL, Perkin Elmer instruments, Courtaboeuf, France) and equipped with a Molecular Sieve 0.5-nm column (3 m×3.2 mm i.d; Interchim, Montiuc̨on, France) maintained at 50° C. for SF₆, or fitted with a flame ionization detector and equipped with a Porapak N 80-100 mesh column (3 m×3.2 mm i.d.; Alltech France SARL, Templemars, France) maintained at 40° C. for CH₄. The flow rate of the carrier gas was 30 mL.min⁻¹ of N₂ for the SF₆ and 40 mL.min⁻¹ of Helium for the CH₄. Chromatographic analyses were performed after calibration with standard gases (Air Liquide, Mitry-Mory, France) for SF₆ (55 and 195 pg/g) and CH₄ (100 μg/g).

### Rumen Fermentation Study During Wk 4

Rumen sample collection and treatments before analysis During the last wk of each experimental period (wk 4), ruminal content samples (200 g) were taken from the ventral sac of the rumen through the cannula, before and 3 h after the morning feeding. The ruminal pH was immediately measured with a portable pH-meter (CG840, electrode Ag/AgCl, Schott Geräte, Hofheim, Germany). The samples were then treated for fermentation and microbial parameters measurements as follows: 30 g of ruminal content were immediately taken to the laboratory for enzymes extraction from the solid-adherent microbial population (SAM) under anaerobic conditions. An aliquot of 10 g of ruminal content was homogenized in ice using a Polytron grinding mill (Kinematica GmbH, Steinhofhalde Switzerland) at speed 5, for two 1 min cycles with 1 min rest between cycles. Subsequently 2 aliquots of 1.5 g were stored at −80° C. until DNA extraction for bacterial quantification using qPCR. For ruminal fermentation characteristics, 100 g of ruminal content were strained through a polyester monofilament fabric (250 μm mesh aperture) and the filtrate was used for analysis of VFA, lactic acid, ammonia and protozoa counting. For VFA, aliquot of 0.8 mL of fresh rumen juice was mixed with 0.5 mL of a 0.5 N HCl solution containing 0.2% (w/v) metaphosphoric acid and 0.4% (w/v) crotonic acid. For NH₃-N, 5 mL of ruminal filtrate were mixed with 0.5 ml of 5% H₃PO₄. The filtrates were stored at −20° C. until analysis. For protozoa, 3 ml of the fresh filtrate was mixed with 3 ml of methyl green, formalin and saline solution (MFS) and preserved from light until counting. For each sampling time, the ruminal contents were dried at 103° C. during 24 h for DM determination.

### Measurements

Ruminal fermentations Volatile fatty acids and lactate concentrations were respectively determined by gas chromatography (CP 9002 Gas Chromatography, Chrompack, Middelburg, Germany) and an enzymatic method (Enzyplus EZA 891+, D/L-Lactic Acid, Raisio Diagnostics, Rome, Italy) as described in Lettat et al. (2010). For NH₃-N, 5 ml of ruminal filtrate were mixed with 0.5 mL of 5% H₃PO₄ and stored at −20° C. until analysis. Thawed samples were centrifuged at 10,000 g for 10 min and NH₃-N concentration was determined in the supernatant using the Berthelot reaction (Park et al., 2009). The reaction was carried out in duplicate in 96-well plates using Infinity M200 spectrophotometer (Tecan Austria GmbH, Grödig, Austria).

Protozoa counting Protozoa were enumerated in a Dolfuss cell (Elvetec Services, Clermont-Ferrand, France) according to the method of Jouany and Senaud (1983).

Polysaccharidase activities of solid-associated microorganisms

Polysaccharidase activities involved in the degradation of plant cell wall (EC 3.2.1.4-cellulase and EC 3.2.1.8-endo-1,4-betaxylanase xylanase) and starch (EC 3.2.1.1-α-amylase) were determined from the solid-associated microorganisms (SAM) as already described (Lettat et al., 2010; Martin et Michalet-Doreau, 1995). Briefly, 30 g of solid phase were washed with 350 ml anaerobic MES buffer (pH 6.5, 39° C.) to remove the non-associated and loosely-associated microbial populations, and then recovered by filtration (100 μm). A sample (5 g) of washed digesta containing the SAM was out to under anaerobic environment then suspended in 25 mL of anaerobic MES buffer and stored at −80° C. pending enzyme extraction. SAM fraction was disrupted by defrosting and ultrasonic disintegration (four 30-s periods with 30-s intervals at 4° C.; Sonicator S-400, Misonix Inc., Farmingdale, N.Y., USA). The enzymes were recovered by centrifugation (15 000 g, 15 min, 4° C.), and the supernatant was stored in capped tubes at −80° C. before assay. Polysaccharidase activities were determined by assaying the amount of reducing sugars released from purified substrates (birchwood xylan, carboxymethyl cellulose and potato starch) after 1 h at 39° C. Briefly, the reducing sugars were converted into colored product using PAHBAH (4-hydroxybenzhydrazide) in the presence of bismuth and quantified spectrophotometrically at 410 nm (Lever, 1977). The protein content of the enzyme preparations was determined according to Pierce and Suelter (Pierce et Suelter, 1977) using bovine serum albumin as standard in 96 well plates using Infinity M200 spectrophotometer (Tecan Austria GmbH, Grödig, Austria). Enzyme activities were expressed as μmol of reducing sugar released per mg protein per hour.

Bacterial quantification by qPCR The DNA extraction was performed using the Fast DNA Spin Kit and purification was done with the Gene Clean turbo Kit (MP Biomedicals, Ilkirch, France) according to the manufacturer instructions. Briefly, 250 mg of frozen milled ruminal contents were weighed into the provided sterile tube containing silica beads and lysis buffer. Lysis of bacteria was performed using a Precellys 24 apparatus (Bertin Technology, France). The yield and the purity of the extracted nucleic acids were assessed by optical density measurement with a Nanoquant Infinite M200 apparatus (Tecan Austria GmbH, Grödig, Austria) using a dedicated quantification plate. Absorbance intensity at 260 nm was used to assess the concentration of nucleic acids in 2 μL of sample, while absorbance ratios 260/280 and 260/230 were used to check sample purity (protein and salts contamination, respectively). Quantitative PCR (qPCR) was carried out using AB StepOne Plus System (Applied Biosystem, Courtaboeuf, France). Detection was based on SYBR green chemistry. The reaction mix contained 1xSYBR Premix Ex Taq (Lonza Verviers SPRL, Verviers, Belgium), 0.5 μM of each forward and reverse primer and 2 μL of genomic DNA at a concentration of 40 ng/μL. Each reaction was fun in triplicate in a final volume of 20 μL in 96-well plates (Applied Biosystem, Courtaboeuf, France). During this study, we quantified total bacteria, Prevotella genus, Fibrobacter succinogenes, Ruminococcus albus, Ruminococcus flavefaciens, Streptococcus bovis, Lactobacillus genus, Megasphaera elsdenii and meihanogen Archaea using specific primers that target the rrs genes for which sequences and amplification programs are summarized in Tables 2 and 3. For this study we chose to realize an absolute quantification using specific 16S rDNA standards from R. flavefaciens c94 (ATCC 19208), R. albus 7 (ATCC 27210), F. succinogenes S85 (ATCC 19169), S. bovis (DSM 20480), P. bryantii B14 (DSM 11371), M. elsdenii (DSMZ 20460), Lb. acidophilus and Methanobrevibacter smithii (DSM 861). Results of enumeration of each species are expressed as % of total bacteria/g DM of rumen content.

### Statistical Procedure

All data were analyzed in repeated time using the MIXED procedure of SAS with SP(POW) as covariance structure. Within each latin square, the period (1 to 4), treatment (C vs. P, vs. Lp+P, vs. Lr+P), time (−1 vs. +3 h; only for rumen characteristics), and treatment×time interactions were considered as fixed effect, and animal as random. Results were considered significant for P-value<0.05 and trends were discussed at 0.05<P<0.1. As no effect of DFM treatment was observed before feeding (−1 h) only data after feeding (+3 h) will be presented and discussed below.

### RESULTS AND DISCUSSION

We aimed to induce two SARA situations in dairy cows using diets that differ in the amount and rate of degradation of their carbohydrates. The HSD was used to induce a propionic SARA whereas butyric SARA was expected using the LSD. Accordingly to our hypothesis that DFMs mode of action to prevent SARA depends on the ruminal fermentation patterns, we compared their effectiveness to regulate ruminal fermentations under the two situations induced. We used the definition proposed by Sauvant et al. (1999) which considers a mean ruminal pH of 6.25 as SARA threshold because it corresponds to a meantime of 4 h where pH is between 5.8 and 6.25 (Sauvant et al., 1999). Thus, according to this definition SARA has been successfully induced using the two diets (mean pH of 5.73 and 5.94 for the ASD and LSD, respectively).

### The Rumen Fermentation Study

DFMs effects on ruminal fermentations and microbial parameters are presented on Table 4 and 5. With the HSD that was used to induce propionic SARA situation, DFMs increased both minimum and mean ruminal pH by +0.41 and +0.24 pH units on average, respectively. This advantageous effect on pH was associated with a concomitant reduction in lactate concentrations (Table 4). No effect was observed on total VFA but cows that received P and Lr+P favored propionate production at the expense of acetate and/or butyrate as shown by the acetate propionate ratios that reached 1.55 and 1.69 (P<0.05), respectively. For cows supplemented with Lp+P, ruminal fermentations were similar to the two other treatment but changes were not significant (P>0.1). Supplementing cows with the 3 DFMs decreased or tended to decrease methanogens population. No other effect was observed on the microbial population except trends for increase in total bacteria and R. albus proportion in cows supplemented with Lp+P and Lr+P, respectively. In cows fed the LSD, ruminal fermentation parameters were not affected by DFMs supplementation except a decline in lactate concentration for cows treated with P (P=0.04). This same treatment tended to increase total bacteria (P=0.06). Lp+P increased amylase and cellulase activities (P=0.05) and tended to improve xylanase activity (P<0.1). Whilst not wishing to be bound by any theory we think that DFMs did not alter ruminal pH and fermentations because the ruminal environment was not acidotic enough as it was the case with the cows fed the HSD diet.

### The Animal Performances Study

The results of supplementing dairy cows fed either the HSD or the LSD with Propionibacterium P63 alone or combined with Lb. plantarum 115 or Lb. rhamnosus 32 on intake, milk yield and composition are shown in Table 6.

Diet digestibility Total tract digestibility of DM, OM, NDF, ADF, hemicellulose and starch are presented in Table 7. On the HSD, cows supplemented with P tended to increase hemicellulose digestion (+6.71%, P=0.08). By contrast, DFMs effects were more pronounced on the LSD when the propionibacteria and lactobacilli combinations were fed. Indeed, Lp+P supplement improved DM and OM digestion by 2.48 and 2.15% (P<0.05) respectively, and tended to increase hemicellulose digestion by 9.31 % (P=0.07). NDF and ADF digestibilities were also improved by 6.00 and 4.31% (P<0.05) respectively in cows fed Lr+P. These beneficial effects on digestibility, especially with the LSD, may be related to the increase in total bacteria, as well as to the enzymatic activities improvement, especially for cows supplemented with Lp+P.

Methane production Daily methane emissions are presented in Table 8. Loss of GE intake as eructed methane averaged 4.1 and 5.9% for cows fed the HSD and LSD by producing 207 and 288 g/d of CH₄, respectively. Cows fed the HSD produced similar amounts of methane for all treatments; except for those supplemented with Lp+P that produced approximately 20% less methane compared to control cows. On the LSD, cows supplemented with Lr+P depressed methane production by 25% on average (P<0.05) whatever the expression units used. Because ruminal fermentation and microbial parameters as well as intake and milk production remained similar among treatments, we cannot easily explain how Lr+P depressed methane production. However, whilst not wishing to be bound by any theory this effect could be related to the fiber digestibility improvement observed for this same treatment. Our invention is the first to demonstrate efficient methane mitigation in ruminant without negative effects on ruminal fermentations and animal performances using bacterial DFMs. However, mechanisms by which Lb. rhamnosus 32 plus Propionibacterium P63 act remain to be elucidated.

In conclusion, our study shows that propionibacteria and lactobacilli-based DFMs effectiveness in dairy cows is conditioned by ruminal fermentation patterns. During propionic SARA, the three DFMs mitigated pH decline by increasing propionate production that reduces the hydrogen available for protons formation. Moreover, an improvement in the ruminal buffering capacity may have accounted for that. By contrast, in cows fed the LSD, using both P63 and the two lactobacilli strains was more effective than P63 alone. Indeed, supplementing cows with Lp+P increased fiber digestibility which can be related to fibrolytic enzymes activities improvement, whereas Lr+P increased fiber digestibility and reduced methane production. Based on these original results, the DFMs evaluated in this work could be useful to prevent SARA or mitigate methane outputs in dairy cows.

All publications mentioned in the above specification are herein incorporated by reference. Various modification and variations of the described methods and compositions of the present invention will be apparent to those skilled in the art without departing from the scope and spirit of the present invention. Although the present invention has been described in connection with specific preferred embodiments. It should be understood that the invention as claimed should not be unduly limited to such specific embodiments. Indeed, various modifications of the described modes for carrying out the invention which are obvious to those skilled in agriculture, biochemistry, microbiology and molecular biology or related fields are intended to be within the scope of the following claims.

### REFERENCES

AFNOR. 1985. Aliments des animaux. Méthodes d'analyses françaises et communautaires. Dosage de l'amidon. Pages 123-125 in Méthode polarimétrique, 2nd ed. Assoc. Fr. Normalisation, Paris, France.

AOAC. 1990. Official Methods of Analysis. 14th ed. Assoc. Off. Anal. Chem., Arlington, Va.

AOAC. 1997. Official Methods of Analysis, 16th ed. Assoc. Off. Anal. Chem, Gaithersburg, Md.

Beauchemin, K. A., W. Z. Yang, D. P. Morgavi, G. R. Ghorbani, W. Kautz, and J. A. Leedle. 2003. Effects of bacterial direct-fed microbials and yeast on site and extent of digestion, blood chemistry, and subclinical ruminal acidosis in feedlot cattle. J. Anim. Sci. 81: 1628-1640.

INRA. 1989. Ruminant Nutrition: Recommended Allowances and Feed Tables, INRA Editions, Paris, France.

Johnson, K. A., H. H. Westberg, J. J. Michal, and M. W. Cossalman. 2007. The SF6 tracer technique: Methane measurement from ruminants. In: H. P. S. Makkar and P. E. Vercoe (eds.) Measuring Methane Production From Ruminants. p 33-67 Springer Netherlands.

Jouany, J.-P., and J. Senaud. 1983. Influence des ciliés du rumen sur l'utilisation digestive de différents régimes riches en glucides solubles et sur les produits terminaux formés dans le rumen. II.—Régimes contenant de l'inuline, du saccharose et du lactose. Reprod. Nutr. Dev., 23:607-623.

Lettat, A., P. Noziere, M. Silberberg, D. P. Morgavi, C. Berger, and C. Martin, 2010. Experimental feed induction of ruminal lactic, propionic, or butyric acidosis in sheep, J. Anim. Sci. 88: 3041-3046.

Martin, C., and B Michalet-Doreau. 1995. Variations in mass and enzyme activity of rumen microorganisms: Effect of barley and buffer supplements. J. Sci. Food Agric. 67: 407-413.

Martin, C., J. Rouel, J. P. Jouany, M. Doreau, and Y. Chilliard. 2008. Methane output and diet digestibility in response to feeding dairy cows crude linseed, extruded linseed, or linseed oil. J. Anim. Sci. 86: 2642-2650.

Mathieu, F., J. Jouany., J. Sénaud, J, Bohatier, G. Bertin, and M. Mercier. 1996. The effect of Saccharomyces cerevisiae and Aspergillus oryzrae on fermentations in the rumen of faunated and defaunated sheep; protozoal and probiotic interactions. Reprod. Nutr. Dev. 36: 271-287.

Park, G., H. Oh, and S. Ahn. 2009. Improvement of the ammonia analysis by the phenate method in water and wastewater. Bull. Korean Chem. Soc. 30:2032-2038.

Pierce, J., and C. H. Suelter. 1977. An evaluation of the Coomassie brilliant blue G-250 dye-binding method for quantitative protein determination. Anal. Biochem. 81: 478-480.

Sauvant, D., F. Meschy, and D. Mertens. 1999. Components of ruminal acidosis and acidogenic effects of diets. INRA Prod. Anim. 12: 49-60.

Van Soest, P. J., J. B. Robertson, and B. A. Lewis. 1991. Methods for dietary fiber, neutral detergent fiber, and nonstarch polysaccharides in relation to animal nutrition. J: Dairy Sci. 74: 3583-3597.

TABLE 1
Ingredient and chemical composition of the experimental diets

| | High Starch Diet | Low Starch Diet |
|---|---|---|
| | (HSD) | (LSD) |
| Ingredients, % DM | | |
| Corn silage | 44 | —¹ |
| Grass silage | — | 55 |
| Hay | 11 | — |
| Grain mix² | 35.3 | — |
| Soybean meal | 8.7 | 8 |
| Urea | 1 | — |
| Citrus pulp | — | 12 |
| Dehydrated beet pulp | — | 20 |
| Molasses, beet | — | 5 |
| Mineral-vitamin premix (kg/d)³ | 0.25 | 0.25 |
| Chemical composition⁴ | | |
| UFL/kg DM⁵ | 0.99 | 0.96 |
| PDIE, g/kg DM⁶ | 109 | 106 |
| OM, % of DM | 90.28 | 88.74 |
| CP, % of DM | 14.84 | 13.53 |
| NDF, % of DM | 36.41 | 36.95 |
| ADF, % of DM | 22.02 | 27.35 |
| Starch, % of DM | 38.17 | 2.23 |
| Ether extract, % of DM | 2.21 | 2.66 |
| GE, MJ/kg of DM | 16.85 | 16.93 |

¹Ingredient not included.
²Composition: barley (14%), wheat (11%) and corn (9%) agglomerated with 1% cane molasses.
³Composition (g/kg); P, 2.5; Ca, 20; Mg, 4.5; Na, 3.5 (Galaphos Midi Duo GR, CCPA, Aurillac, France).
⁴Chemical analysis was performed on 4 samples per period for silages and hay but on pooled samples for concentrates.
⁵Feed Unit for Lactation (INRA, 1989).
⁶Protein digested in the small intestine supplied by rumen-undegraded dietary protein and by microbial protein from rumen-fermented OM (INRA, 1989).

TABLE 2
16S rDNA based primers used for qPCR, quantification of bacteria and Archaea

| Target organism | Primer set | Primer sequences 5′-3′ | Source |
|---|---|---|---|
| All bacteria | 520 F | AGC AGC CGC GGT AAT | Edwards et al., |
| | 799 R2 | CAG GGT ATC TAA TCC TGT T | 2008 |
| F. succinogenes | FibSuc3F | GCG GGT AGC AAA CAG GAT TAG A | |
| | FibSuc3R | CCC CCG GAC ACC CAG TAT | |
| R. albus | RumAlb3F | TGT TAA CAG AGG GAA GCA AAG CA | |
| | RumAlb3R | TGC AGC CTA CAA TCC GAA CTA A | |
| R. flavefaciens | Runfla3F | TGG CGG ACG GGT GAG TAA | Stevenson and |
| | RumFla3R | TTA CCA TCC GTT TCC AGA AGC T | Weimer, 2007 |
| Genus Prevotella | PrevGen4F | GGT TCT GAG AGG AAG GTC CCC | |
| | PrevGen4R | TCC TGC ACG CTA CTT GGC TG | |
| S. bovis | StrBev2F | TTC CTA GAG ATA GGA AGT TTC TTC GG | |
| | StrBov2R | ATG ATG GCA ACT AAC AAT AGG GGT | |
| M. elsdenii | MegEls1F | GAG CGA AAC TGC GAT GCT AGA | Khafipour et al., |
| | MegEls1R | CGC CTC AGC GTC AGT TGT C | 2009 |
| Genus | Lacto 05F | AGC AGT AGG GAA TCT TCC A | Furet at al., |
| Lactobacillus | lacto 04R | CGC CAC TGG TGT TCY TCC ATA TA | 2009 |
| Methenogen | 1174F | GAG GAA GGA GTG GAC GAC GGTA | Mosoni et al., 2011; |
| Archaea | 1406-1389R | ACG GGC GGT GTG TGC AAG | Ohene-Adjei et al., |
| | | | 2007 |

TABLE 3
Summary of the qPCR programs used for bacteria and Archaea quantification

| | | 40 cycles | | |
|---|---|---|---|---|
| Target | Initial heating | Denaturation | Annealing/Elongation | Dissociation curve |
| All bacteria | | | | |
| F. succinogenes | | | | |
| R. albus | | 95° C., 15 sec | 60° C., 30 sec | temperature increased by |
| R. flavefaciens | 95° C., 10 sec | | | +0.3° C./min from 60 to |
| Genus Prevotella | | | | 95° C. |
| S. bovis | | | 63.9° C., 30 sec | |
| M. elsdenii | | 95° C., 10 sec | 64.9° C., 15 sec than 72° C., 15 sec | |
| Methanogen Archaea | | 96° C., 15 sec | 63° C., 15 sec than 72° C., 30 sec | |
| Genus Lactobacillus | 96° C., 10 min | 97° C., 30 sec | 60° C., 1 min | |

TABLE 4
Effects of DFMs supplementation on the ruminal fermentation characteristics in dairy cows fed two different acidotic diets.

| High Starch Diet (HSD) | | | | | | | | Low Starch Diet (LSD) | | | | | | | |
|---|---|---|---|---|---|---|---|---|---|---|---|---|---|---|---|
| | | | | | P-value² | | | | | | | | P-value | | |
| | DFMs¹ | | | | (DFMs vs. C) | | | | DFMs | | | | (DFMs vs. C) | | |
| | | Lp + | Lr + | | | Lp + | Lr + | | | Lp + | Lr + | | | Lp + | Lr + |
| C | P | P | P | SEM | P | P | P | C | P | P | P | SEM | P | P | P |

Ruminal fermentation characteristics

| | | | | | | | | | | | | | | | | |
|---|---|---|---|---|---|---|---|---|---|---|---|---|---|---|---|---|
| Ruminal pH | | | | | | | | | | | | | | | | |
| Mean | 5.73 | 5.96 | 6.01 | 5.93 | 0.07 | 0.02 | 0.004 | 0.03 | 5.94 | 5.94 | 6.04 | 6.08 | 0.08 | 0.98 | 0.36 | 0.20 |
| Minimum | 5.34 | 5.73 | 5.85 | 5.67 | 0.12 | 0.03 | 0.01 | 0.06 | 5.73 | 5.77 | 5.83 | 5.87 | 0.12 | 0.78 | 0.51 | 0.35 |
| Total VFA, | 141.4 | 145.4 | 135.9 | 150.9 | 0.46 | 0.68 | 0.57 | 0.34 | 144.2 | 148.4 | 146.6 | 137.6 | 5.63 | 0.6 | 0.76 | 0.4 |
| mM | | | | | | | | | | | | | | | | |
| Acetate, % | 56.63 | 51.91 | 53.99 | 51.83 | 1.83 | 0.06 | 0.27 | 0.05 | 62.05 | 62.1 | 61.91 | 62.35 | 0.79 | 0.96 | 0.9 | 0.79 |
| Propionate, % | 26.03 | 33.76 | 29.39 | 31.61 | 2.46 | 0.02 | 0.29 | 0.08 | 17.99 | 18.21 | 18.69 | 17.78 | 0.35 | 0.66 | 0.17 | 0.69 |
| Butyrate, % | 12.19 | 9.18 | 11.45 | 11.54 | 1.19 | 0.07 | 0.65 | 0.69 | 15.06 | 14.65 | 14.57 | 15 | 0.56 | 0.59 | 0.52 | 0.94 |
| Minor VFA³, | 5.16 | 5.15 | 5.17 | 5.03 | 0.38 | 0.09 | 0.99 | 0.81 | 4.91 | 5.05 | 4.83 | 4.87 | 0.28 | 0.73 | 0.84 | 0.92 |
| % | | | | | | | | | | | | | | | | |
| A:P ratio | 2.33 | 1.55 | 2.06 | 1.69 | 0.29 | 0.03 | 0.44 | 0.08 | 3.46 | 3.43 | 3.33 | 3.53 | 0.1 | 0.83 | 0.36 | 0.66 |
| Lactate, mM | 3.68 | 1.15 | 2.24 | 1.76 | 0.55 | 0.01 | 0.07 | 0.02 | 1.96 | 0.80 | 1.37 | 2.08 | 0.37 | 0.04 | 0.26 | 0.81 |
| NH₃—N, mM | 16.15 | 9.49 | 11.61 | 8.9 | 3.55 | 0.2 | 0.38 | 0.17 | 10.99 | 10.15 | 8.9 | 8.45 | 2.04 | 0.77 | 0.48 | 0.39 |
| Ethanol, mM | 4.49 | 3.92 | 4.83 | 5.41 | 7.49 | 0.38 | 0.61 | 0.17 | 4.14 | 4.29 | 4.15 | 5.52 | 0.63 | 0.87 | 0.99 | 0.14 |

¹DFMs: direct-fed microbials.
²Effect of each DFM vs. contol cows (C).
³Minor VFA: sum of iso-butyrate, iso-valerate, valerate and caproate.

TABLE 5
Effects of DFMs supplementation on enzyme activities and microbial
composition in dairy cows fed two different acidotic diets

| High Starch Diet (HSD) | | | | | | | |
|---|---|---|---|---|---|---|---|
| | DFMs¹ | | | | P-value² (DFM vs. C) | | |
| C | P | Lp + P | Lr + P | SEM | P | Lp + P | Lr + P |

Specific polysaccharidase activities³, μmol of reducing sugar/mg protein/hour

| | | | | | | | | |
|---|---|---|---|---|---|---|---|---|
| Amylase | 28.8 | 35.72 | 26.26 | 44.08 | 14.92 | 0.74 | 0.90 | 0.46 |
| Xylanase | 14.94 | 5.45 | 6.13 | 16.39 | 5.72 | 0.25 | 0.29 | 0.86 |
| Cellulase | 0.4 | 0.52 | 0.79 | 1.53 | 1.19 | 0.94 | 0.80 | 0.46 |

Rumen bacteria, ns copies/g DM of rumen content

| | | | | | | | | |
|---|---|---|---|---|---|---|---|---|
| Total bacteria, log₁₀ | 12.49 | 12.65 | 12.69 | 12.55 | 0.08 | 0.18 | 0.10 | 0.61 |
| R. albus, %⁴ | 0.34 | 0.77 | 0.98 | 1.41 | 0.34 | 0.33 | 0.17 | 0.04 |
| R. flavefaciens, % | 0.43 | 0.50 | 0.39 | 0.42 | 0.18 | 0.79 | 0.88 | 0.99 |
| F. succinogenes, % | 1.10 | 0.90 | 1.02 | 1.15 | 0.18 | 0.46 | 0.76 | 0.86 |
| S. bovis, % | 0.002 | 0.001 | 0.002 | 0.001 | 0.0009 | 0.56 | 0.84 | 0.56 |
| Lactobacillus spp., % | 0.12 | 0.23 | 0.19 | 0.17 | 0.06 | 0.13 | 0.28 | 0.50 |
| Prevotella spp., % | 43.95 | 51.08 | 47.74 | 49.73 | 4.33 | 0.14 | 0.40 | 0.22 |
| M. eisdenil, % | 0.47 | 0.93 | 0.38 | 1.46 | 0.50 | 0.53 | 0.91 | 0.21 |
| Archaea, % | 1.05 | 0.64 | 0.71 | 0.78 | 0.15 | 0.03 | 0.06 | 0.10 |

Rumen protozoa, 10⁴/mL of rumen fluid

| Protozoa⁵ | 7.69 | 0.43 | 6.60 | 0.84 | 4.96 | 0.23 | 0.85 | 0.25 |
|---|---|---|---|---|---|---|---|---|
| | Low Starch Diet (LSD) | | | | | | | |
| | | DFMs | | | | P-value (DFMs vs. C) | | |
| | C | P | Lp + P | Lr + P | SEM | P | Lp + P | Lr + P |

Specific polysaccharidase activities³, μmol of reducing sugar/mg protein/hour

| | | | | | | | | |
|---|---|---|---|---|---|---|---|---|
| Amylase | 0.56 | 0.61 | 3.55 | 1.38 | 1.01 | 0.97 | 0.05 | 0.57 |
| Xylanase | 9.18 | 7.85 | 22.7 | 6.49 | 6.09 | 0.86 | 0.09 | 0.73 |
| Cellulase | 1.06 | 1.31 | 6.04 | 1.45 | 1.83 | 0.92 | 0.05 | 0.87 |

Rumen bacteria, ns copies/g DM of rumen content

| | | | | | | | | |
|---|---|---|---|---|---|---|---|---|
| Total bacteria, log₁₀ | 12.3 | 12.42 | 12.38 | 12.35 | 0.04 | 0.06 | 0.17 | 0.40 |
| R. albus, %⁴ | 0.11 | 0.27 | 0.23 | 0.21 | 0.07 | 0.15 | 0.26 | 0.37 |
| R. flavefaciens, % | 0.23 | 0.13 | 0.27 | 0.32 | 0.06 | 0.31 | 0.70 | 0.38 |
| F. succinogenes, % | 0.72 | 0.88 | 0.74 | 0.70 | 0.15 | 0.47 | 0.94 | 0.89 |
| S. bovis, % | 0.0008 | 0.0007 | 0.0002 | 0.0003 | 0.0003 | 0.93 | 0.27 | 0.33 |
| Lactobacillus spp., % | 0.028 | 0.032 | 0.046 | 0.034 | 0.01 | 0.79 | 0.25 | 0.67 |
| Prevotella spp., % | 37.36 | 34.69 | 43.15 | 41.24 | 3.64 | 0.60 | 0.28 | 0.45 |
| M. eisdenil, % | 0.26 | 0.11 | 0.52 | 0.21 | 0.18 | 0.57 | 0.33 | 0.87 |
| Archaea, % | 0.82 | 0.82 | 0.65 | 0.67 | 0.12 | 0.99 | 0.28 | 0.36 |

Rumen protozoa, 10⁴/mL of rumen fluid

| | | | | | | | | |
|---|---|---|---|---|---|---|---|---|
| Protozoa⁵ | 20.51 | 25.75 | 21.06 | 21.32 | 2.6 | 0.17 | 0.88 | 0.82 |

¹DFMs: direct-fed microbials.
²Effect of each DFM vs. control cows (C).
³Amylase, cellulase and xylanase activities were determined with starch, cellulose and hemicellulose, respectively.
⁴Bacteria species and Archaea expressed as proportion of total bacteria
⁵Protozoa: sum of small (<100 μm) and big (>100 μm) Entodinium, Isotrocha and Dasytricha.

TABLE 6
Effects of DFMs supplementation on intake and milk production
and composition in dairy cows fed two different acidotic diets

| | High Starch Diet (HSD) | | | | | | | |
|---|---|---|---|---|---|---|---|---|
| | | DFMs¹ | | | | P-value² (DFM vs. C) | | |
| | C | P | Lp + P | Lr + P | SEM | P | Lp + P | Lr + P |
| Intake, kg/d | | | | | | | | |
| DM | 18.53 | 19.19 | 18.29 | 19.6 | 0.99 | 0.59 | 0.84 | 0.38 |
| OM | 17.30 | 17.93 | 17.06 | 18.32 | 1.00 | 0.59 | 0.83 | 0.38 |
| ADF | 3.77 | 3.92 | 3.69 | 3.97 | 0.21 | 0.55 | 0.77 | 0.43 |
| NDF | 6.26 | 6.51 | 6.14 | 6.61 | 0.35 | 0.56 | 0.78 | 0.42 |
| Hemicellulose | 2.49 | 2.59 | 2.45 | 2.63 | 0.14 | 0.57 | 0.79 | 0.41 |
| Starch intake | 7.14 | 7.30 | 7.25 | 7.68 | 0.35 | 0.67 | 0.77 | 0.18 |
| GE intake, MJ/d | 295 | 306 | 291 | 312 | 16.26 | 0.58 | 0.61 | 0.39 |
| Milk production, kg/d | | | | | | | | |
| Milk yield | 25.03 | 25.66 | 24.20 | 26.36 | 1.71 | 0.67 | 0.58 | 0.37 |
| 4% FCM³ | 24.42 | 24.10 | 23.66 | 25.37 | 2.17 | 0.86 | 0.68 | 0.59 |
| Milk composition, | | | | | | | | |
| g/kg | | | | | | | | |
| Fat yield | 38.24 | 37.70 | 36.47 | 37.41 | 3.44 | 0.91 | 0.73 | 0.87 |
| Protein yield | 31.39 | 31.69 | 31.64 | 29.24 | 1.27 | 0.86 | 0.88 | 0.24 |
| Lactose yield | 51.60 | 52.01 | 51.42 | 51.81 | 0.76 | 0.6 | 0.82 | 0.78 |
| SCC⁴ × 10³ mL | 317 | 605 | 142 | 57 | 255 | 0.42 | 0.62 | 0.46 |
| | Low Starch Diet (LSD) | | | | | | | |
| | | DFMs | | | | P-value (DFMs vs. C) | | |
| | C | P | Lp + P | Lr + P | SEM | P | Lp + P | Lr + P |
| Intake, kg/d | | | | | | | | |
| DM | 18.81 | 18.02 | 18.58 | 18.31 | 1.25 | 0.46 | 0.83 | 0.62 |
| OM | 16.10 | 15.41 | 15.86 | 15.64 | 1.07 | 0.45 | 0.79 | 0.60 |
| ADF | 4.84 | 4.62 | 4.77 | 4.70 | 0.33 | 0.44 | 0.82 | 0.61 |
| NDF | 6.55 | 6.26 | 6.46 | 6.37 | 0.44 | 0.44 | 0.81 | 0.62 |
| Hemicellulose | 1.711 | 1.64 | 1.68 | 1.67 | 0.11 | 0.45 | 0.79 | 0.64 |
| Starch intake | 0.32 | 0.25 | 0.30 | 0.29 | 0.05 | 0.34 | 0.73 | 0.59 |
| GE intake, MJ/d | 300 | 287 | 296 | 292 | 20.30 | 0.45 | 0.80 | 0.61 |
| Milk production, kg/d | | | | | | | | |
| Milk yield | 23.63 | 22.64 | 22.66 | 23.15 | 1.38 | 0.18 | 0.19 | 0.47 |
| 4% FCM³ | 22.58 | 23.58 | 22.71 | 23.39 | 1.61 | 0.41 | 0.91 | 0.49 |
| Milk composition, | | | | | | | | |
| g/kg | | | | | | | | |
| Fat yield | 37.20 | 43.6 | 39.97 | 40.62 | 2.85 | 0.16 | 0.52 | 0.43 |
| Protein yield | 27.35 | 29.26 | 29.42 | 28.82 | 1.43 | 0.38 | 0.34 | 0.49 |
| Lactose yield | 51.74 | 51.8 | 52.17 | 52.71 | 0.58 | 0.93 | 0.53 | 0.18 |
| SCC⁴ × 10³ mL | 183 | 85 | 397 | 47 | 163 | 0.68 | 0.39 | 0.58 |

¹DFMs: direct-fed microbials.
²Effect of each DFM vs. control cows (C).
³FCM = Fact corrected Milk
⁴SCC = Somatic Cell Count

TABLE 7
Effects of DFMs supplementation on total tract digestibility of DM, OM, fibers and starch in dairy cows fed two different acidotic diets

| | High Starch Diet (HSD) | | | | | | | | Low Starch Diet (LSD) | | | | | | | |
|---|---|---|---|---|---|---|---|---|---|---|---|---|---|---|---|---|
| | | DFMs¹ | | | | P-value² (DFM vs. C) | | | | DFMs | | | | P-value (DFMs vs. C) | | |
| | | | Lp + | | | | Lp + | | | | Lp + | | | | Lp + | |
| | C | P | P | Lr + P | SEM | P | P | Lr + P | C | P | P | Lr + P | SEM | P | P | Lr + P |
| DM, % | 69.79 | 70.91 | 69.31 | 69.86 | 0.71 | 0.28 | 0.63 | 0.93 | 67.48 | 67.86 | 69.94 | 68.30 | 0.93 | 0.70 | 0.04 | 0.40 |
| OM, % | 72.208 | 73.28 | 71.81 | 72.18 | 0.72 | 0.26 | 0.67 | 0.99 | 72.20 | 72.4 | 74.35 | 72.88 | 0.71 | 0.77 | 0.02 | 0.34 |
| ADF, % | 46.34 | 47.94 | 46.58 | 44.15 | 2.39 | 0.65 | 0.94 | 0.54 | 48.45 | 51.43 | 49.1 | 54.45 | 3.40 | 0.38 | 0.84 | 0.01 |
| NDF, % | 43.81 | 47.46 | 42.79 | 41.76 | 1.76 | 0.19 | 0.70 | 0.44 | 46.08 | 46.94 | 48.79 | 50.39 | 2.19 | 0.60 | 0.11 | 0.02 |
| Hemicellose, % | 40.01 | 46.72 | 37.04 | 38.14 | 2.29 | 0.08 | 0.40 | 0.59 | 39.22 | 33.61 | 48.63 | 38.96 | 2.99 | 0.23 | 0.07 | 0.95 |
| Starch, % | 98.18 | 98.01 | 98.35 | 98.32 | 0.39 | 0.62 | 0.62 | 0.69 | 100 | 88.45 | 84.45 | 86.41 | 21.10 | 0.20 | 0.17 | 0.18 |

¹DFMs: direct-fed microbials.
²Effect of each DFM vs. control cows (C).

TABLE 8
Effects of DFMs supplementation on methane emissions
in dairy cows fed two different acidotic diets.

| | High Starch Diet (HSD) | | | | | | | |
|---|---|---|---|---|---|---|---|---|
| | | DFMs¹ | | | | P-value² (DFMs vs. C) | | |
| | C | P | Lp + P | Lr + P | SEM | P | Lp + P | Lr + P |
| CH4, g/d | 206.64 | 227.36 | 157.60 | 236.26 | 37.05 | 0.61 | 0.25 | 0.46 |
| CH4, g/kg DMI | 11.05 | 11.99 | 8.69 | 12.15 | 1.63 | 0.60 | 0.21 | 0.53 |
| CH4, g/kg OM intake | 11.83 | 12.84 | 9.32 | 13.00 | 1.75 | 0.60 | 0.22 | 0.53 |
| CH4, g/kg NDF intake | 32.69 | 35.56 | 25.90 | 36.09 | 4.83 | 0.59 | 0.23 | 0.51 |
| CH4, g/kg ADF intake | 54.36 | 59.19 | 43.13 | 60.06 | 7.80 | 0.59 | 0.23 | 0.51 |
| CH4, g/kg of digested DM | 16.76 | 17.68 | 13.14 | 18.36 | 2.44 | 0.72 | 0.19 | 0.53 |
| CH4, g/kg of digested OM | 16.40 | 17.54 | 12.99 | 18.04 | 2.40 | 0.66 | 0.21 | 0.51 |
| CH4, g/kg of digested NDF | 75.36 | 76.77 | 61.47 | 89.30 | 11.41 | 0.91 | 0.31 | 0.30 |
| CH4, g/kg of digested ADF | 117.91 | 128.59 | 95.47 | 143.04 | 19.39 | 0.66 | 0.36 | 0.30 |
| CH4, g/kg of milk | 8.26 | 8.75 | 6.49 | 9.04 | 1.10 | 0.69 | 0.19 | 0.52 |
| CH4, g/kg of 4% FCM³ | 8.45 | 9.35 | 6.75 | 9.53 | 1.19 | 0.59 | 0.33 | 0.52 |
| CH4, g/kg of GE intake | 4.14 | 4.49 | 3.27 | 4.55 | 0.61 | 0.60 | 0.22 | 0.53 |
| | Low Starch Diet (LSD) | | | | | | | |
| | | DFMs | | | | P-value (DFMs vs. C) | | |
| | C | P | Lp + P | Lr + P | SEM | P | Lp + P | Lr + P |
| CH4, g/d | 315.27 | 302.08 | 301.54 | 236.28 | 22.17 | 0.57 | 0.56 | 0.01 |
| CH4, g/kg DMI | 17.03 | 17.13 | 16.05 | 13.04 | 1.21 | 0.95 | 0.51 | 0.03 |
| CH4, g/kg OM intake | 19.91 | 20.04 | 18.80 | 15.26 | 1.42 | 0.94 | 0.52 | 0.03 |
| CH4, g/kg NDF intake | 48.95 | 49.32 | 46.19 | 37.55 | 3.47 | 0.93 | 0.51 | 0.03 |
| CH4, g/kg ADF intake | 66.31 | 66.83 | 62.57 | 51.07 | 4.65 | 0.93 | 0.51 | 0.03 |
| CH4, g/kg of digested DM | 26.90 | 26.60 | 24.27 | 20.36 | 2.04 | 0.90 | 0.30 | 0.03 |
| CH4, g/kg of digested OM | 27.73 | 27.66 | 25.34 | 21.10 | 2.03 | 0.98 | 0.34 | 0.03 |
| CH4, g/kg of digested NDF | 109.96 | 103.65 | 96.92 | 76.60 | 9.38 | 0.48 | 0.17 | 0.01 |
| CH4, g/kg of digested ADF | 141.73 | 129.04 | 131.20 | 96.28 | 11.68 | 0.19 | 0.26 | 0.001 |
| CH4, g/kg of milk | 13.61 | 13.63 | 13.24 | 10.26 | 1.16 | 0.98 | 0.69 | 0.01 |
| CH4, g/kg of 4% FCM³ | 14.20 | 13.00 | 13.44 | 10.13 | 1.23 | 0.21 | 0.41 | 0.003 |
| CH4, g/kg of GE intake | 6.37 | 6.42 | 6.01 | 4.89 | 0.45 | 0.93 | 0.62 | 0.03 |

¹DFMs: direct-fed microbials.
²Effect of each DFM vs. control cows (C).
³FCM = Fact corrected milk

1. A method for reducing methane production in a ruminant animal comprising the step of administering to said ruminant animal an effective amount of at least one strain of bacterium of the genus Propionibacterium.

2. The method of claim 1, wherein said strain of bacterium belongs to the species Propionibacterium jensenii, Propionibacterium acidipropionici, Propionibacterium freudenreichii or Propionibacterium freudenreichii ssp shermanii.

3. The method of claim 2 wherein said strain belongs to the species Propionibacterium jensenii.

4. The method of claim 3 wherein the strain is Propionibacterium jensenii P63.

5. The method of any preceding claim, further comprising the step of administering to said ruminant animal an effective amount of at least one strain of bacterium of the genus Lactobacillus.

6. The method of claim 5, wherein said strain of bacterium of the genus Lactobacillus belongs to the species L. paracasei, L. casei, L. acidophilus, L. buchnerii, L. farciminis, L. rhamnosus, L. reuteri, L. fermentum, L. brevis, L. lactis, L. plantarum, L. sakei, L. salviarium, L. helveticus, L. amylovorus, L. curvatus, L. cellobiosus. L. amylolyticus, L. alimentanus, L. aviaries, L. crispatus, L. curvatus, L. gallinarum, L. hilgardii, L. johnsonii, L. kefiranofaecium, L. kefiri, L. mucosae, L. panis, L. pentosus, L. pontis, L. zeae or L. sanfranciscensis.

7. The method of claim 6 wherein the strain of bacterium of the genus Lactobacillus belongs to the species L. plantarum or L. rhamnosus.

8. The method of claim 7 wherein the strain of the bacterium of the genus Lactobacillus is L. plantarum Lp115 or L. rhamnosus.Lr32.

9. The method of any one of claims 5 to 8 wherein the at least one strain of bacterium of the genus Propionibacterium and the at least one strain of the bacterium of the genus Lactobacillus are administered as a mixture.

10. The method of any one of claims 5 to 9 wherein said mixture of at least two strains of bacteria is a mixture of at least one strain of L. plantarum or L. rhamnosus and at least one strain of Propionibacterium jensenii.

11. The method of claim 10, wherein said mixture is a mixture of L. plantarum Lp115 or L. rhamnosus Lr32 and Propionibacterium jensenii P63.

12. The method of any preceding claim, wherein the at least one strain of bacteria is inactivated.

13. The method of any preceding claim, wherein said effective amount of at least one strain of bacterium is administered to said ruminant animal by supplementing food intended for said animal with said effective amount of at least one strain of bacterium.

14. The method of any preceding claim, wherein the method additionally improves the digestibility of the supplementing food.

15. The method of any preceding claim wherein the method additionally increases milk fat production by the ruminant animal.

18. The method of any preceding claim wherein the method additionally increases milk lactose production by the ruminant animal.

17. The method of any preceding claim wherein the method additionally improves the body weight of the ruminant animal.

18. The method of any preceding claim, wherein said ruminant animal is selected from the members of the Ruminantia and Tylopoda suborders.

19. The method of any preceding claim, wherein said ruminant animal is selected from the members of the Antilocapridae, Bovidae, Cervidae, Giraffidae, Moschidae, Tragulidae families.

20. The method of claim 19, wherein said ruminant animal is a cattle, goat, sheep, girafee, bison, yak, water buffalo, deer, camel, alpaca, llama, wildebeest, antelope, pronghorn or nilgai.

21. The method of claim 20, wherein said ruminant animal is a cattle or sheep.

22. The method of claim 21, wherein said ruminant animal is a cattle.

23. A feed supplement for a ruminant animal for reducing methane production comprising at least one strain of bacterium the genus Propionibacterium.

24. The feed supplement of claim 23, comprising at least one strain of bacterium belonging to the species Propionibacterium jensenii, Propionibacterium acidipropionici, Propionibacterium freudenreichii and Propionibacterium freudenreichii ssp shermanii.

25. The feed supplement of claim 23 or 24 further comprising at least one strain of bacterium of the genus Lactobacillus.

26. The feed supplement of claim 25, comprising at least one strain of bacterium belonging to the species L. paracasei, L. casei, L. acidophilus, L. buchnerii, L. farciminis, L. rhamnosus, L. reuteri, L. fermentum, L. brevis, L. lactis, L. plantarum, L. sakei, L. salviarium, L. helveticus, L. amylovorus, L. curvatus, L. cellobiosus, L. amylolyticus, L. alimentarius, L. aviaries, L. crispatus, L. curvatus, L. gallinarum, L. hilgardii, L. johnsonii, L. kefiranofaecium, L. kefiri, L. mucosae, L. panis, L. pentosus, L. pontis, L. zeae or L. sanfranciscensis.

27. The feed supplement of claim 26 or 26 comprising at least one strain of L. plantarum or L. rhamnosus and at least one strain of Propionibacterium jensenii.

28. The feed supplement of claims 27 comprising at least one of L. plantarum Lp115 or L. rhamnosus Lr32 and Propionibacterium jensenii P63.

29. A feed for a ruminant animal, wherein said feed is supplemented with a feed supplement according to any one of claims 24 to 28.

30. A method for reducing methane production by a ruminant animal, said method comprising the step of administering to said animal a feed supplement according to any one of claims 24 to 28.

31. A method for increasing milk fat production by a ruminant animal, said method comprising the step of administering to said animal a feed supplement according to any one of claims 24 to 28.

32. A method for increasing milk lactose production by a ruminant animal, said method comprising the step of administering to said animal a feed supplement according to any one of claims 24 to 28.

33. A method for increasing the body weight of a ruminant animal, said method comprising the step of administering to said animal a feed supplement according to any one of claims 24 to 28.
